(19) United States
(12) Patent Application Publication
(10) Pub. No.: US 2022/0021467 A1
(43) Pub. Date: Jan. 20, 2022

(54) Ultrasonic Communication Phased Array

(71) Applicant: Nuvasive Specialized Orthopedics, Inc., San Diego, CA (US)

(72) Inventors: Youngsam Bae, Aliso Viejo, CA (US); Everett Van Zuiden, Chula Vista, CA (US); Khoa Pham, Garden Grove, CA (US)

(21) Appl. No.: 17/358,755

(22) Filed: Jun. 25, 2021

Related U.S. Application Data
(60) Provisional application No. 63/051,892, filed on Jul. 15, 2020.

(51) Int. Cl. H04B 11/00; A61B 5/00

(52) U.S. Cl. CPC H04B 11/00; A61B 5/0031; A61B 5/0028

(57) ABSTRACT

The present disclosure provides an external transceiver configured for ultrasonic communication with a medical implant, the external transceiver including an array of ultrasonic transducers configured to be placed adjacent to a patient's skin and each ultrasonic transducer of the array of ultrasonic transducers configured to send and receive an ultrasonic signal.

### CROSS REFERENCE TO RELATED APPLICATIONS

The present application claims the benefit under 35 U.S.C. § 119(e) of U.S. provisional patent application No. 63/051,892, filed Jul. 15, 2020. The entirety of the foregoing application is incorporated by reference as though fully set forth herein.

### FIELD OF DISCLOSURE

The present disclosure pertains to the field medical devices. More specifically, the present disclosure pertains to medical devices configured to transmit data transcutaneously using an ultrasound signal.

### BACKGROUND

Medical implants have various forces exerted on them in vivo, especially medical implants that are adjustable in situ. Such adjustable medical implants for example, are used in limb lengthening and spinal adjustable surgical procedures to treat conditions such as limb deformities and scoliosis. Typically, these adjustable medical implants are secured to one or more bones and gradually adjusted over time until some desired patient outcome is achieved.

These surgical implants and procedures do not include an accurate and non-invasive means of measurement of in vivo conditions, such as forces and pressures, present at the implant site. Particularly, after the implant is implanted and during the course of treatment. What is needed is some kind of device and method to perform needed measurements of conditions present at the implant site non-invasively.

Further, these surgical implants and procedures do not include reliable transcutaneous communication devices or methods to achieve bidirectional communication of power/data between implants and other medical devices.

### SUMMARY OF THE INVENTION

The present disclosure provides transcutaneous ultrasonic communication between medical devices located on and/or within a body of a patient.

In some aspects, the present disclosure provides an implant, the implant having an ultrasonic transducer, wherein the ultrasonic transducer is configured to send and receive ultrasonic signal.

In some aspects, the present disclosure provides an external transceiver configured for ultrasonic communication with a medical implant, the external transceiver including an array of ultrasonic transducers configured to be placed adjacent to a patient's skin and each ultrasonic transducer of the array of ultrasonic transducers configured to send and receive an ultrasonic signal.

In some aspects, the present disclosure provides a system the system including: and implant having an ultrasonic transducer configured to send and receive ultrasonic signals and an external transceiver configured for ultrasonic communication with the implant, the external transceiver including an array of ultrasonic transducers configured to be placed adjacent to a patient's skin and each ultrasonic transducer of the array of ultrasonic transducers configured to send and receive ultrasonic signals.

In some aspects, the present disclosure provides a method for ultrasonic communication, the method comprising the steps: implanting an implant within a patient, the implant comprising an ultrasonic transducer configured to transmit and receive ultrasonic signals; and providing adjacent to the patient's skin an external transceiver configured to communicate with the implant using an ultrasound signal, the external transceiver comprising: an array of ultrasonic transducers configured to transmit and receive ultrasonic signals.

In some aspects, the method may include transmitting an ultrasound signal to the implant using the external transceiver, the ultrasound signal configured to activate the implant

In some aspects, the phased array may be configured to set an azimuthal focal point and steer the azimuthal focal point relative to the phased array.

In some aspects, the method may include rasterizing the azimuthal focal point to maximize an amount of reception of ultrasonic signals transmitted to the implant.

In some aspects, the method may include rasterizing a focal depth of the azimuthal focal point to maximize reception of an ultrasonic signal transmitted to the implant.

In some aspects, the method may include varying a focal width of the azimuthal focal point to maximize reception of an ultrasonic signal transmitted to the implant.

### BRIEF DESCRIPTION OF THE DRAWINGS

These and other features will be further understood by those with skill in the art upon a review of the appended drawings, wherein:

FIG. 1 shows an implant in accordance with a first embodiment, the implant located within a body of a patient and configured to transcutaneously send and receive ultrasonic signals from an external transceiver;

FIG. 2 shows an implant in accordance with a second embodiment, the implant located within a body of a patient and configured for transcutaneous bidirectional ultrasonic data communication;

FIG. 3A shows a side view of an implant in accordance with a third embodiment, the implant configured for transcutaneous bi-directional ultrasonic data communication;

FIG. 3B shows a cross-sectional side view of the implant in accordance with the third embodiment, the implant shown having a sensor module disposed therein, the sensor module configured for transcutaneous bi-directional ultrasonic communication;

FIG. 4 shows a schematic of ultrasonic communication between an implant and an external transceiver;

FIG. 5 shows a phased array of ultrasonic transducers changing an angle of propagation of ultrasound waves relative thereto;

FIG. 6 shows an isocontour plot of intensity of an ultrasound signal transmitted by a phased array focused to an azimuthal focal point;

FIG. 7 shows an axial position vs. intensity graph of the ultrasound signal from FIG. 6;

FIG. 8 shows an axial position vs. intensity graph for an ultrasound signal transmitted by a phased array focused to an azimuthal focal point of 25 mm;

FIG. 9 shows an axial position vs. intensity graph for an ultrasound signal transmitted by a phased array focused to an azimuthal focal point of 50 mm;

FIG. 10 shows an axial position vs. intensity graph for an ultrasound signal transmitted by a phased array focused to an azimuthal focal point of 75 mm;

FIG. 11 shows an axial position vs. intensity graph for an ultrasound signal transmitted by a phased array focused to an azimuthal focal point of 100 mm;

FIG. 12 shows an isocontour plot of intensity of a signal transmitted by a phased array focused to an azimuthal focal point displaced by 0 mm;

FIG. 13 shows an isocontour plot of intensity of an ultrasound signal transmitted by a phased array focused to an azimuthal focal point laterally displaced by 15 mm;

FIG. 14 shows an isocontour plot of intensity of an ultrasound signal transmitted by a phased array focused to an azimuthal focal point laterally displaced by 30 mm;

FIG. 15 shows a plot of 2D contours of multiple azimuthal focal points at various locations relative to a phased array;

FIG. 16 shows a lateral displacement of an azimuthal focal point;

FIG. 17 shows a change in focal depth of an azimuthal focal point;

FIG. 18 shows an external transceiver transmitting an unfocused activation signal to an implant disposed somewhere within a body of a patient;

FIG. 19 shows the implant responding to the external transceiver;

FIG. 20 shows the external transceiver transmitting a focused communication signal to the implant with the implant shown paired in communication therewith; and

FIG. 21 shows a phased array in communication with an implant disposed within a bone of a patient.

### DETAILED DESCRIPTION OF THE PREFERRED EMBODIMENTS

For purposes of explanation and not limitation, details and descriptions of certain preferred embodiments are hereinafter provided such that one having ordinary skill in the art may be enabled to make and use the invention. These details and descriptions are representative only of certain preferred embodiments, however, and a myriad of other embodiments which will not be expressly described will be readily understood by those having skill in the art upon a thorough review hereof. Accordingly, any reviewer of the instant disclosure should interpret the scope of the invention by the claims, and such scope shall not be limited by the embodiments described and illustrated herein.

Bidirectional ultrasonic communication in medical implants can provide power, enhanced control, and biofeedback between implants and other medical devices.

Similar to Radio Frequency (RF) signals, which utilize light in the RF band, information may be conveyed within the body using ultrasound waves and ultrasound signals via known amplitude and phase shifting techniques. Phase-Shift Keying is a digital modulation process which conveys data by changing the phase of a constant frequency carrier wave. The modulation is accomplished by varying the sine and cosine inputs at a precise time. It is widely used for wireless LANs, RFID and BLUETOOTH. Binary phase-shift keying (BPSK) or any modulation technique may be used in ultrasound communication including: On-Off Keying (OOK), Amplitude-Shift Keying (ASK) and Frequency-Shift Keying (FSK).

The frequency of ultrasound sound waves chosen to establish the bidirectional ultrasonic communication in implants may be in any frequency of ultrasound, but are generally greater than 20 kilohertz. In some embodiments, the frequency of ultrasound sound waves may be between 200 and 400 kilohertz, for example: about 300 kilohertz. The benefits of utilizing ultrasound sound waves for power and/or data transmission in implants include: (1) that ultrasound sound waves have both favorable propagation and minimal attenuation characteristics through metal or solid mediums (e.g., metallic medical implants), and (2) that ultrasound sound waves transmit data transcutaneously through various aqueous tissues in animals (e.g. human skin, muscle and bone).

Once a bidirectional ultrasound communication link is established, the implant may have a power consumption of between 0.5 mW and 80 mW, 1 mW and 60 mW, and 2.0 mW and 40 mW, 10 mW, 5 mW, and any subrange thereof. The ultrasound transducer may consume about 20 mW of power when in operation. The transducer may be configured to transmit data through at least four inches of water or aqueous tissues at a rate of 5 values per second (1 kb/s) with a data reliability of 95%. Data reliability transmitted from the transducer at these power levels may be at least 95%, at least 98%, at least 99%, at least 99.9%, or 100%. “Data reliability” means reliability over 10 minutes as calculated from a bit error rate (BER).

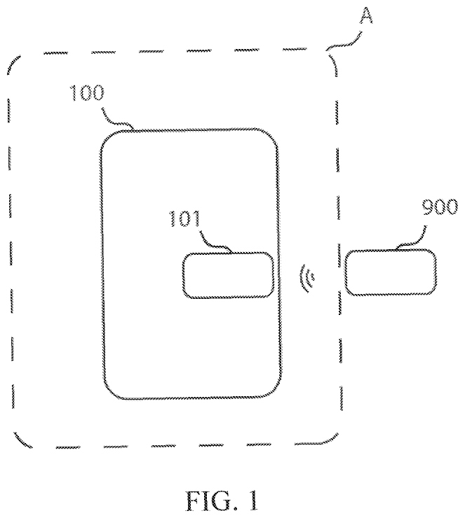

Turning to the drawings, in FIG. 1 a schematic diagram is provided showing an implant 100 configured to receive wireless power via an ultrasound signal. The implant 100 is shown disposed within a body of a patient A. Patient A may be any animal including a human. The implant 100 includes at least one ultrasonic transducer 101 configured to receive the ultrasonic signal sent by an external transceiver 900, and convert that ultrasonic signal to electrical energy. The ultrasonic transducer 101 may be for example a piezoelectric transducer, and the piezoelectric transducer may be operably connected to other circuitry within the implant 100. The electrical energy harvested by the ultrasonic transducer 101 may be used to activate and power any circuitry disposed within the implant 100. Similarly, the implant 100 may include a power storage device. For example: one or more of a battery and capacitor.

The implant 100 may be, by way of example: an adjustable implant, a distraction rod, an intramedullary rod, an expandable intervertebral cage, and any other implant or medical device intended for placement on and within the body of a patient. In some embodiments, wireless activation and or powering of the implant 100 using ultrasonic waves, may eliminate a need for internal power storage devices disposed within various implants.

The implant 100, may be made of polyether ether ketone (PEEK), polyetherketone (PEK), titanium (Ti), and any other material known and used to make implants. Including any biocompatible thermoplastic and metallic materials. Preference may be given to materials with known favorable ultrasonic transmission characteristics. The implant may be fabricated using known fabrication techniques for the materials chosen including for example: additive manufacturing, welding, bonding, and molding techniques. The implant may be dimensioned in accordance with a treatment plan. And the implant may include other electronic components and circuitry, for example: to operably couple the ultrasonic transducer 101 to a controller.

The ultrasonic transducer 101 may include any device that induces sound waves or mechanical vibration in the ultrasound spectrum, including for example: a piezoelectric transducer, a single crystal ultrasonic transducer, a lead zirconate titanate (PZT) ultrasonic transducer, piezoelectric polyvinylidene fluoride (PVDF) ultrasonic transducer, capacitive micromachined ultrasonic transducers (CMUT), piezoelectric micromachined ultrasonic transducers (CMUT), and any ultrasonic transducer known and used.

In some embodiments, the external transceiver 900 may retrieve an ID tag of the implant 100 using an ultrasound signal. The implant may include an integrated circuit and an ultrasonic transceiver 101, which are used to transmit data to the external transceiver 900 using an ultrasound signal. In some embodiments, the external transceiver may obtain an ID tag by providing power to the implant 100 using an ultrasound signal wherein the implant 100 harvests enough energy from the ultrasound signal to enable the implant 100 to transmit the ID tag via an ultrasonic signal transmitted back to the external transceiver 900. In some embodiments, the ID tag is obtained through a coupled state, for example an LC circuit. In some embodiments, enough energy is harvested to activate the implant 100 and establish a bidirectional communication link between the implant 100 and the external transceiver 900 using ultrasound signals.

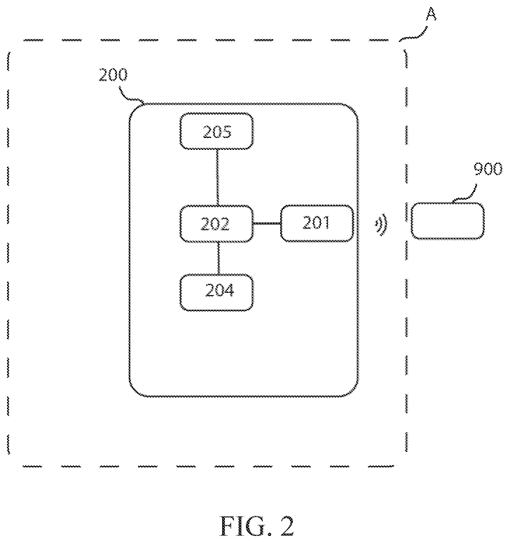

Turning to FIG. 2, a schematic diagram is provided showing an implant 200 in accordance with a second embodiment, the implant 200 configured for transcutaneous ultrasonic data communication with at least an external transceiver 900. The implant 200 is shown having operatively connected circuitry including at least one ultrasonic transducer 201, a controller 202, a sensor 205, and a power storage device 204.

The controller 202 may be any type of controller 202 known and used in the art including: high performance microcontrollers (MCUs), Programmable System on Chip (PSoC), Application Specific Integrated Circuit (ASIC) and any other type of controller and microcomputer. The controller 202 may be disposed on a printed circuit board which may also contain other electronic circuitry and connect other electrical components including: Analog to Digital Converter (ADC), Digital to Analog Converter (DAC), op-amps, memory, phase shifters, and any other electrical component. The controller may further include a frequency synthesizer (i.e., creates carrier waves for ultrasonic transducer 201), power amplifiers and noise filters (i.e., conditions carrier wave), power and read strain gauge (i.e., force sensor controls), and may be configured to adjust carrier waves, power, etc., such as by computer executable instructions that interface with a user via a graphical user interface.

A power storage device 204 may be provided. The power storage device 204 may include a battery, a capacitor, and any other electronic charge or power storage device. The power storage device 204 may include a rechargeable battery (e.g. Lithium ion rechargeable battery). The power storage device 204 may include a solid state battery and any battery having any known mechanism or battery chemistry.

The implant 200 may include a charging circuit operably connected to the power storage device 204 and the piezoelectric transducer 201. The charging circuit may be integrated into one or more of the controller 202 and the printed circuit board. The charging circuit may include a digital switch wherein upon receiving an ultrasonic signal modulated at an activation frequency or charging frequency the electronic switch is configured to enable charging of the power storage device with electrical energy harvested by the ultrasonic transducer. The power storage device 204 may be operably connected to the controller 202 via any electronic conductor including wires, boards, and interconnects. Other details of the charging circuit may include details found in any charging circuit commonly known and used in the art.

In other embodiments, other known wireless charging circuits and techniques including inductive coupling and magnetic coupling may be used to wirelessly transfer power to the implant 200.

In some embodiments, an external transceiver 900 may activate the circuitry of the implant 200 by transmitting an ultrasound signal to the ultrasonic transducer 201. The ultrasound signal may be received by the ultrasonic transducer 201 and converted into electrical energy. The controller 202 may be programmed such that upon receipt of an ultrasound signal corresponding to a particular modulated signal, for example a particular step function of a particular temperance, the controller 202 will open/close an electrical switch and activate the device and place the implant 200 in an awake state. Similarly, in other embodiments a particular step function may be used to open/close an electrical switch and deactivate the device from the awake state to conserve power stored in the power storage device 204.

In some embodiments, the controller 202 may be programmed to time out after a certain period of time, for example if the piezoelectric transducer 201 has not sent or received ultrasonic signals for a set period of time.

In some embodiments, the controller 202 may be programmed to turn off the power storage device 204 or to put the implant 200 to sleep for a certain period of time to conserve power. For example, the controller may activate the implant 200 to transmit ultrasonic signal for ¼ of 1 second. During this ¼ of the second the implant 200 is said to be active or in awake state. The controller may deactivate the implant 200 for ¾ of the second. During this ¾ of the second the device is said to be deactivated or in a sleep state.

In some embodiments the implant 200 may include one or more sensors 205 operably connected to the controller 202. The sensors 205 may be designed to measure temperature, force, pressure, capacitance, resistance, or be any other physical property or characteristic of the implant 200 or measure information indicative of a biological condition from surrounding anatomical structures of the patient A. The sensor may include a position sensor (e.g. optical sensor), a force sensor, or any known sensor. In the instant embodiment the sensor 205 may be configured to sense force for example.

In some embodiments, the sensor 205 may include a Micro-Electro-Mechanical-System (MEMS) sensor. These sensors provide a reduced profile (e.g. 1 μm-100 μm size). The MEMS sensor may include an accelerometer, pressure sensor, gas sensors, humidity sensor, a gyrosensor, ambient light sensor, optical sensor, gesture sensor, proximity sensor, touch sensor, or any other mechanical element or sensory functionality.

The sensor 205 may communicate a sensor reading to the controller 202, which may convert the reading to a modulated electrical signal. The modulated electrical signal may then be used to drive the ultrasonic transducer 201, which then transmits an ultrasonic signal at a frequency corresponding to the modulated electrical signal.

The controller 202 may change analogue information from the sensor 205 to digital values and may drive modulation of the ultrasonic transducer 201, to transmit data using ultrasound waves.

The implant 200 may be any type of implant including an adjustable implant. The adjustable implant may include any actuator known and used in the art. As one with skill in the art may appreciate, the actuator may be an electric motor and the implant may be configured to harvest ultrasonic waves transmitted by another implant or an external transceiver, and convert the ultrasonic waves to electrical energy to power the actuator. In some embodiments, closed loop control of the implant 200 may be achieved using the ultrasound signal to relay information between two or more of an implant 200, an external transceiver 900 and an external adjustment device.

Ultrasonic data communication provides a reliable communication link between one or more implants in or near the body, one or more external transceiver, and one or more tertiary devices. An ultrasonic signal can even be used to establish a network of devices placed on or within the body of a patient.

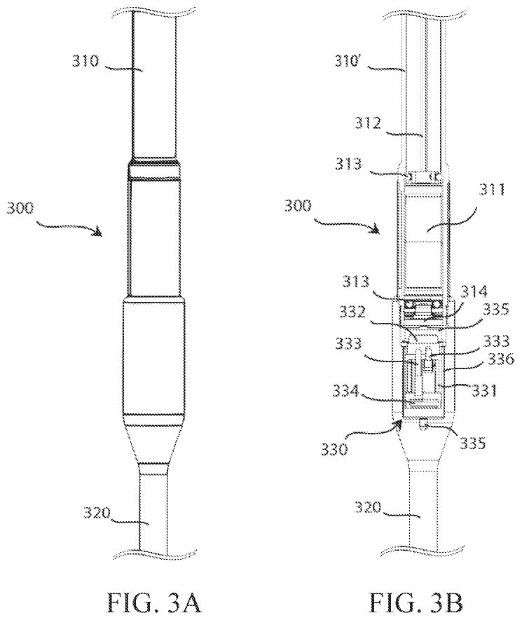

In FIG. 3A-3B, an implant 300 including a distraction rod is shown. The implant 300 includes a first portion 310 configured to be attached to a bone of a patient in a first location, a second portion 320 configured to be attached to a bone of a patient in a second location. The implant 300 may be any type of adjustable implant. By way of example, an adjustable implant may include magnetically adjustable systems, such as the PRECICE® or MAGEC® magnetically adjustable implant systems for spinal and limb lengthening procedures sold by NuVasive, Inc. of San Diego, Calif. Such adjustable systems are disclosed in, for example, U.S. Pat. Nos. 9,398,925 and 9,393,117, which are incorporated by reference herein in their entireties.

FIG. 3B shows a cross-sectional view of the implant 300, the first portion 310 includes a distraction rod. The distraction rod comprises a magnet 311, and the magnet 311 is connected to a lead screw 312. Upon an axial rotation of the magnet 311 by an externally applied rotating magnetic field, the lead screw 312 will rotate. Rotation of the lead screw 312 will cause an axial distraction of the distraction rod, and thereby change a dimension of the implant 300. Rotation of the magnet 311 may be achieved using an external remote control which may further include an external transceiver 900 as described below.

Now, implants experience numerous forces in vivo. For example, as the illustrated distraction rod 310 is distracted, axial forces will push down on the magnet 311. Thrust bearings 313 are provided to mitigate the effect of these forces on the rotation of the magnet 311. The thrust bearings 313 transfer load from the lead screw to the housing of the implant. However, when using an external adjustment device to noninvasively adjust an adjustable implant, biofeedback is often limited.

The implant 300 in FIG. 3B includes a sensor module 330 disposed within the second portion 320. The sensor module 330 includes an ultrasonic transducer 331 including for example a tubular piezoelectric transducer operably connected to a controller 332. The ultrasonic transducer 331 is configured to transmit and receive ultrasonic signals. The tubular ultrasonic transducer 331 is operably connected to the controller 332 via an interconnect 333. As discussed above, the controller 332 may be any type of controller 332 known and used in the art including high performance microcontrollers (MCUs), Programmable System on Chip (PSoC), or any other type of controller or microcomputer. The controller 332 may be disposed on a printed circuit board which may also contain other electronic circuitry and components therein including: Analog to Digital Converter (ADC), Digital to Analog Converter (DAC), op-amps, memory and any other electronic circuitry known and used in the art.

A power storage device 334 is provided. As discussed above, the power storage device 334 may include a battery, a capacitor, and any other rechargeable power storage device.

The sensor module 330 may include a recharging circuit operably connected to the power storage device 334 and the ultrasonic transducer 331. The recharging circuit may be integrated into a controller 332 or another printed circuit board. The power storage device 334 may be operably connected to the controller 332 via an interconnect 333.

The sensor module 330 is configured to receive an ultrasonic signal sent by an external transceiver 900, and convert that ultrasonic signal to electrical energy using the ultrasonic transducer 331. The recharging circuit may use the generated electrical energy to charge the power storage device 334.

In some embodiments, an external transceiver 900 may activate the circuitry of the sensor module 330 by transmitting ultrasonic waves to the sensor module 330. The ultrasonic waves are received by the ultrasonic transducer 331 and converted into electrical energy. The controller 332 may be programmed such that upon receipt of ultrasonic waves corresponding to a particular modulated signal, for example a particular step function of particular temperance, the controller may open/close an electrical switch and activate the sensor module 330. Similarly, a second particular step function may open/close the electrical switch and deactivate the sensor module 330 to conserve power.

In some embodiments, the controller 332 may be programmed to time out after a certain period of time, wherein if for example the ultrasonic transducer 331 has not sent or received ultrasonic waves for a test period of time, the sensor module 330 will deactivate to thereby conserve charged power levels of the power storage device 334, extending a battery life thereof.

In some embodiments the sensor module 330 may be configured to have a power consumption of between 0.5 mW and 80 mW, 1 mW and 60 mW, and 2.0 mW and 40 mW, 10 mW, 5 mW, or any subrange thereof. The ultrasonic transducer 331 may consume about 20 mW of power when in operation. The ultrasonic transducer 331 may be configured to transmit data at least four inches through water and aqueous tissue at a rate of 5 values per second (1 kb/s) with a data reliability of 95%. Data reliability transmitted from the ultrasonic transducer 331 at these power levels may be at least 95%, at least 98%, at least 99%, at least 99.9%, or 100%. “Data reliability” means reliability over 10 minutes as calculated from a bit error rate (BER).

In some embodiments the sensor module 330 may include one or more sensors 335 operably connected to the controller 332. The sensors 335 may be designed to measure force, temperature, pressure, capacitance, resistance, or be any other type of sensor commonly known and used in the art. In the instant embodiment the sensor module 330 is configured to sense axial force from the distraction device using a force sensor 335. The force sensor 335 of the sensor module 330 is operably coupled to the distraction rod using an adapter plate 314.

The force sensor 335 communicates a sensor reading to the controller 332, which may convert the reading to one or more of a digital and modulated electrical signal. The modulated electrical signal may then be used to drive the ultrasonic transducer 331, which then transmits ultrasonic waves transcutaneously. These ultrasonic waves may be observable by the external transceiver 900. In some embodiments, forms of modulation may include: on-off keying, amplitude shift keying (ASK), frequency shift keying (FSK), phase shift keying (PSK), analogue frequency modulation, and any other form of modulation commonly known and used for data transmission. Advantageously, signals that are modulated may require less power than non-modulated signals and may be transmitted and received at greater distance from the sensor module 330 than non-modulated signals. Modulated signals may also have a greater accuracy than non-modulated signals.

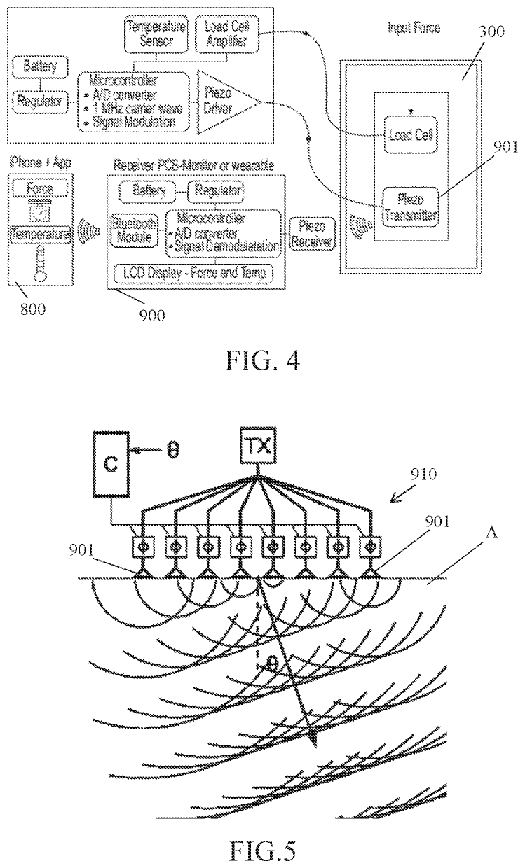

In some embodiments the sensor module 330 includes an encapsulation 336 providing a hermetic seal to the sensor module 330. In order to prevent air gaps or pockets of unnecessary ultrasonic impedance, in some embodiments the piezoelectric transducer 331 is coupled to at least a portion of the encapsulation 336 using a conductive epoxy (see FIG. 4D, 408). In this embodiment the sensor module 330 is disposed adjacent to a surface of the implant 300 to minimize airgaps, reflection, and impedance.

The conductive epoxy may include any conductive material to reduce air gaps, including aluminum epoxy, copper epoxy, copper tape, Ti-epoxy, industry acoustic couplant, or any other material providing favorable electrical and/or acoustic conductive properties. When selecting a conductive epoxy one may consider: i.) impedance matching to improve the ultrasonic transmission efficiency between the implant and the piezoelectric transducer, and ii.) the circuit grounding all of the electronics.

In the instant embodiment, the implant 300 includes a sensor module 330, having various components and features. In some other embodiments, these various components and features may be incorporated directly into the implant 300 similar to those discussed supra. This disclosure is intended to pertain to all variants.

In some embodiments the sensor module 330 may be integrated with a processor circuit of an implant using any type of interconnection, cable, or RF communication protocol. The sensor module 330 may receive data from the processor circuit of the implant, and communicate data transcutaneously to an external transceiver 900.

In some embodiments the external transceiver 900 may obtain data directly from the implant 300, in the instant embodiment data is obtained via the sensor module 330. The external transceiver 900 may then report the data to a tertiary device 800 via an ultrasonic connection, a cable connection, an RF data connection, a wife connection, a bluetooth connection, and any other data communication protocol. The tertiary device may be one or more of a computer, a cell phone, a server, and any other device capable of data communication. The tertiary device may be enabled to drive the external transceiver 900 remotely to activate, communicate with, or control the implant 300 remotely, for example across an internet connection.

FIG. 4 shows an exemplary schematic of ultrasonic communication between an implant 300, an external transceiver 900, and a tertiary device 800.

In the instant embodiment the transceiver 900 includes a piece of wearable technology. The wearable device may be for example: a bracelet, a watch, an arm band, arm sleeve, arm brace, a leg band, a leg sleeve, a leg brace, a back brace, a body sleeve, a neck brace, a head brace, or any type of other wearable device known and used in the art. The wearable device may be made using additive manufacturing techniques including 3D printing.

The tertiary device 800 includes a cellular phone which may be additionally connected to internet and cellular networks. The tertiary device 800 may also include one or more of a personal computer, a smart device, a piece of operating room equipment, and any other electronic device capable of communicating.

The external transducer 900 is configured for ultrasonic communication with medical implants, and includes: an array including two or more ultrasonic transducers 901 configured to be placed adjacent to a patient's skin. Each ultrasonic transducer 901 of the array is configured to send and receive ultrasonic signals. In some embodiments, the external transducer may include a phased array of ultrasonic transducers.

FIG. 5 shows a schematic of an exemplary phased array 910 of ultrasonic transducers 901. It includes an array of two or more ultrasound transducers 901 powered by a transmitter TX. The feed current for each ultrasonic transducer passes through a phase shifter ϕ controlled by a controller C. The curved lines show the wavefronts of sound waves in the ultrasound frequency emitted by each ultrasonic transducer 901 in a body of a patient A. The individual wavefronts are spherical, but they combine (superpose) in front of the ultrasonic transducer 901 to create a plane wave, which is a beam of sound waves travelling in a common direction. The phase shifters ϕ delay the sound waves progressively going up the line so each ultrasonic transducer 901 emits its wavefront later than the one before it. This causes the resulting plane wave to be directed at an angle θ relative to the array's axis. By changing the phase, the controller can instantly change the angle θ of the beam. In some embodiments, the phased array 910 may have a two-dimensional array of transducers instead of the linear array shown here, and similarly the beam may be a surface which can be steered in multiple dimensions.

A phased array 910 provides an ability to steer a direction of beam propagation and an azimuthal focal point 911 as needed to find and/or lock on to an implant 300 configured for ultrasound communication. Maximizing transmission and reception of an ultrasonic data signal between the external transceiver 900 and the implant 300.

The phased array 910 may be a one dimensional as illustrated or a two dimensional array. A one dimensional phased array, for example has multiple ultrasonic transducers 901 disposed in a single column. Each ultrasonic transducer 901 of a one dimensional array is assigned a position relative to their position on the phased array 910 by a controller C configured to interpret signals received by each ultrasonic transducer in the phased array 910 and interpret them relative to their various positions on the phased array 910.

A two dimensional phased array 910 has ultrasonic transducers 901 disposed in for example a matrix. Each ultrasonic transducer 901 may be assigned a location relative to the dimensions of the matrix. For example, the ultrasonic transducers 901 may be arranged in a simple grid, and assigned position values based on their position on the grid. And in some embodiments may be disposed in a circle or ring and assigned position values based on their position using polar coordinates.

Introducing a curvature to the phased array 910 by adding a lens/lensing element one can set an azimuthal focal point 911. As outlined below, by varying the phase of a transmission signal at each ultrasonic transducer one can produce this lensing effect.

Using principles of constructive and destructive wave interference, one can vary the phase of the transmission of an ultrasonic signal for each ultrasonic transducer 901 across the phased array 910 to maximize constructive and destructive interference at an azimuthal focal point thereby maximizing transmission of the ultrasonic signal to that point. As the signal is modulated in amplitude or phase the observed signal will also be modulated and data conveyed. By changing the phase of transmission at various points along the phased array 910 one can effectively steer a lateral position and/or a focal depth of the azimuthal focal point, sometimes called focal point and axial focus, relative to the phased array 910.

Mathematically, wave propagation from a phased array 910 of ultrasonic transducers 901 can be modeled using N-slit diffraction optics, in which the radiation field at the receiving point is the result of the coherent addition of N point sources. Since each individual ultrasonic transducer 901 acts as a slit, emitting sound waves as opposed to light, their diffraction pattern can be calculated by adding the phase shift ϕ to the fringing term.

Beginning from the wave equation describing the N-slit diffraction pattern, with N slits of equal size a and spacing d the wave can be modeled:

$$ψ = {ψ}_{0} \frac{sin ( \frac{ a}{λ} sin θ )}{\frac{ a}{λ} sin θ} \frac{sin ( \frac{N}{2} kd sin θ )}{sin ( \frac{k d}{2} sin θ )}$$

The square of this wave gives us the intensity:

$$I = {{I}_{0} ( \frac{sin ( \frac{ a}{λ} sin θ )}{\frac{ a}{λ} sin θ} )}^{2} {( \frac{sin ( \frac{N}{2} ( \frac{2 d}{λ} sin θ + ϕ ) )}{sin ( \frac{ d}{λ} sin θ + \frac{ϕ}{2} )} )}^{2}$$

$$I = {{I}_{0} ( \frac{sin ( \frac{ a}{λ} sin θ )}{\frac{ a}{λ} sin θ} )}^{2} {( \frac{sin ( \frac{}{λ} Nd sin θ + \frac{N}{2} ϕ )}{sin ( \frac{ d}{λ} sin θ + \frac{ϕ}{2} )} )}^{2}$$

Now, for simplicity, let's assume the ultrasonic transducers are spaced a distance d=λ/4 apart.

$$I = {{I}_{0} ( \frac{sin ( \frac{ a}{λ} sin θ )}{\frac{ a}{λ} sin θ} )}^{2} {( \frac{sin ( \frac{}{4} N sin θ + \frac{N}{2} ϕ )}{sin ( \frac{}{4} sin θ + \frac{ϕ}{2} )} )}^{2}$$

As sine achieves its maximum at π/2, one set the numerator of the second term equal to 1.

$$\frac{}{4} N sin θ + \frac{N}{2} ϕ = \frac{}{2}$$

$$sin θ = ( \frac{}{2} - \frac{N}{2} ϕ ) \frac{4}{N}$$

$$sin θ = \frac{2}{N} - \frac{2 ϕ}{}$$

Thus as N gets large, the term will be dominated by the 2ϕ/π term. As sine can oscillate between −1 and 1, one can see that setting ϕ=−π/2 will send the maximum energy at an angle given by:

$$θ = {sin}^{- 1} \qquad 1 = \frac{}{2} = 90 °$$

Additionally, if one wishes to adjust the angle at which the maximum energy is emitted, one need only to adjust the phase shift ϕ between successive ultrasonic transducers. Indeed, the phase shift corresponds to the negative angle of maximum signal. A similar calculation will show that the denominator is minimized by the same factor.

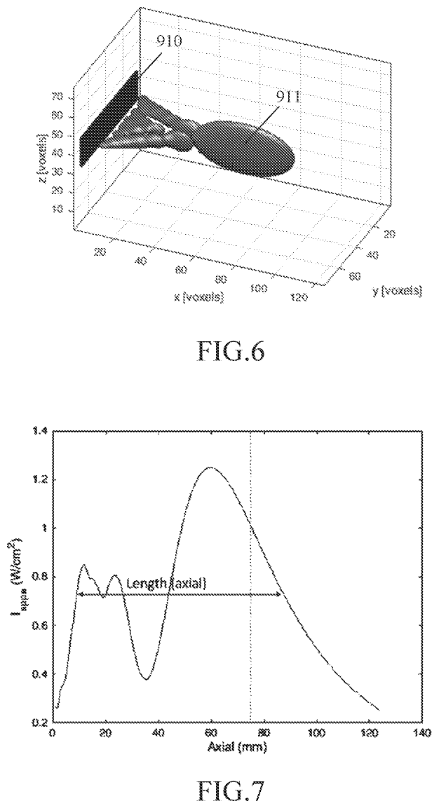

FIG. 6 shows a three-dimensional isocontour representation of average intensity of an ultrasound signal as observed in a three-dimensional space, the ultrasound signal generated from a phased array 910. This isocontour was generated using a phased array 910 including thirty-two ultrasonic transducers 901 focused to an azimuthal focal point 911 having a focal depth of 60 mm and an azimuth displacement of 0 mm. For reference, FIG. 7 shows an axial intensity plot of the beam, with peak intensity observed at the azimuthal focal point 911 of 60 mm.

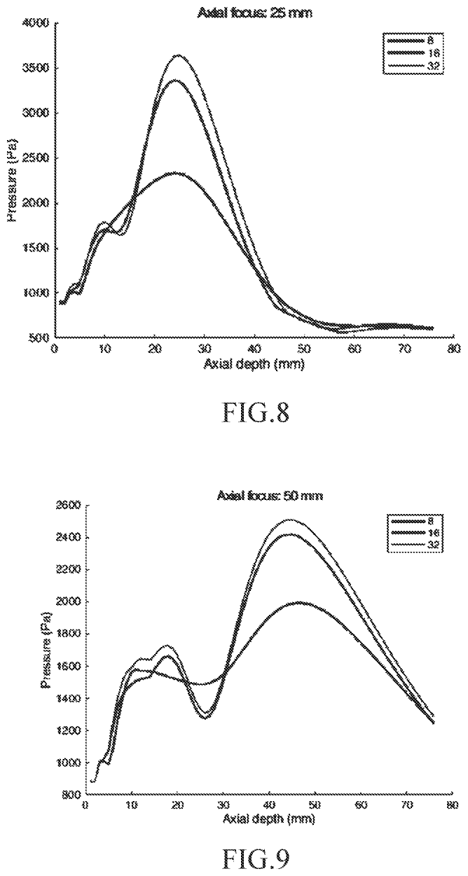

To demonstrate the ability of the phased array 910 to vary the focal depth, FIG. 8-FIG. 11 show intensity plots generated using phased arrays 910 of eight, sixteen, and thirty-two ultrasonic transducers 901.

In FIG. 8 the phased arrays 910 were focused to a focal depth of 25 mm. As the number of ultrasonic transducers (N) is increased from eight ultrasonic transducers 901 to sixteen ultrasonic transducers 901, there is a very large increase in the focused peak intensity at the target azimuthal focal point 911. A relatively small difference is observed from sixteen ultrasonic transducers 901 to thirty-two ultrasonic transducers 901 as compared to the difference observed between eight ultrasonic transducers 901 and sixteen ultrasonic transducers 901.

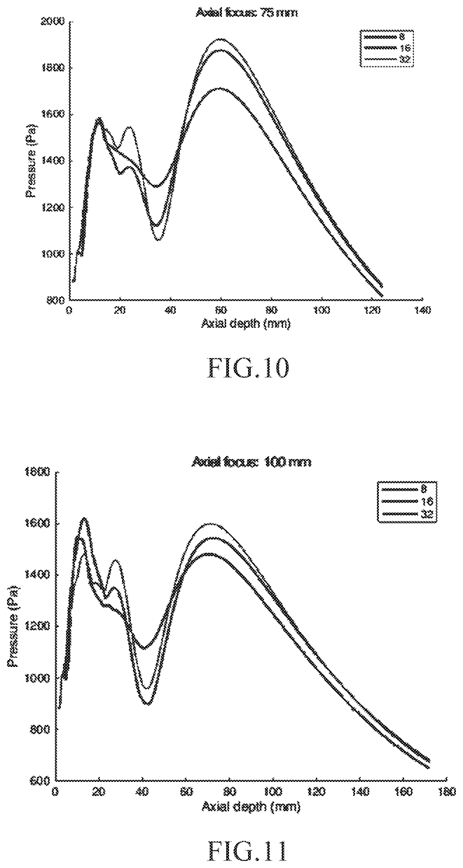

In FIG. 9 the focal depth is increased to 50 mm. In FIG. 10 the focal depth is increased to 75 mm. An in FIG. 11, the focal depth is increased to 100 mm. Observationally, as the focal depth is increased an observed peak intensity at the azimuthal focal point is decreased.

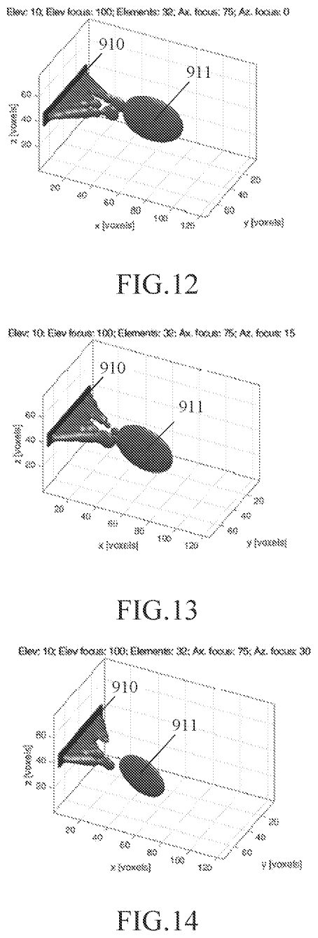

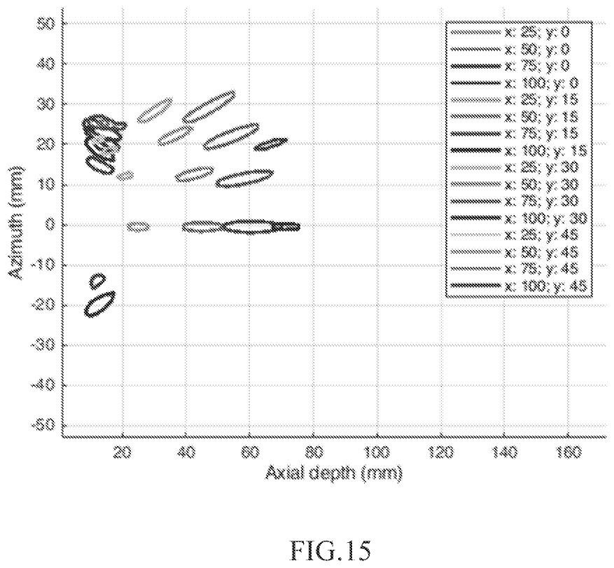

In FIG. 12-FIG. 14 a lateral displacement of the azimuthal focal point is demonstrated. By varying the phase of the signal applied to the ultrasonic transducers 901 of the phased array 910, the azimuthal focal point is shown translated from the center of the ultrasonic array 901 with an azimuth displacement of 0 mm in FIG. 12, to an azimuth displacement of about 15 mm in FIG. 13, and finally to an azimuth displacement of about 30 mm in FIG. 14. Which as one with skill in the art may appreciate, demonstrates a lateral translation of the azimuthal focal point of 30 mm.

FIG. 15 shows some of the experimental azimuthal focal point movements relative to an azimuth and axial depth of the phased array having a number of ultrasonic transducers (N) equal to thirty-two. It is worth noting a symmetrical distribution can be achieved in both the positive and the negative azimuth ranges. Due to the near infinite observable and controllable azimuthal focal points, only a limited number are being illustrated herein but the full range may be achieved and is contemplated herein.

Now, the phased array 910 of ultrasonic transducers 901 may be applied to ultrasound communication in medical implants. An implant 300 configured for ultrasonic communication, such as the intramedullary device 300 of FIG. 3A-FIG. 3B, placed within an intramedullary canal of a bone of a patient A can be paired to an external transceiver 900 having a phased array 910. In some embodiments, the external transceiver 900 may be disposed on or included as part of an external adjustment device configured to control and change a dimension of the implant 300.

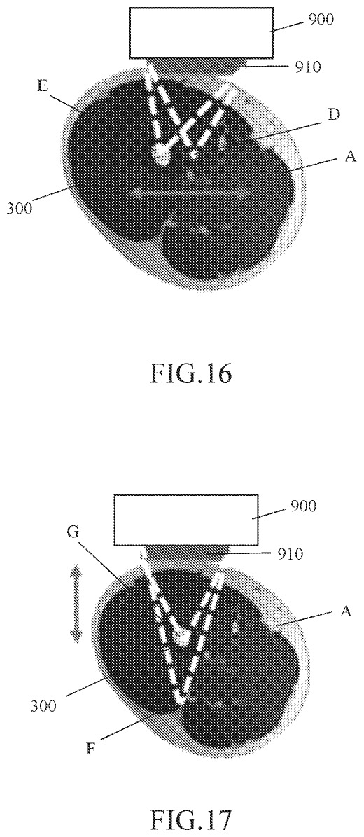

Turning to FIG. 16, the external transceiver 900 includes: a phased array 910 of ultrasonic transducers 901 configured to be placed adjacent to a patient's skin with each ultrasonic transducer 901 of the array configured to send and receive an ultrasonic signal. The phased array 910 is configured to set an azimuthal focal point 911 and steer the azimuthal focal point 911 laterally relative to the phased array 910. Here the phased array 910 is configured to steer the azimuthal focal 911 point laterally, from for example a first location D to a second location E, changing a lateral position of the azimuthal focal point 911 relative to the phased array 910 to align the azimuthal focal point 911 with the implant 300 disposed within the body of the patient A. This helps the external transceiver 900 find the implant 300 and improve ultrasonic signal transmission between the external transceiver 900 and the implant 300. This includes improving data transmission and reliability.

In some embodiments, the phased array 910 is configured to steer the azimuthal focal point 911 and change a focal depth of the azimuthal focal point 911 relative to the phased array 910, as illustrated in FIG. 17. This focal depth may be varied to move the azimuthal focal point 911, from for example a first location F to a second location G, to align the azimuthal focal point 911 with the implant 300 disposed within the body of the patient A. This helps the external transceiver 900 find the implant 300 and improves ultrasonic signal transmission between the external transceiver 900 and the implant 300. This includes improving data transmission and reliability. In some conditions, this may help to increase transmission by directing more energy through areas of low attenuation within the body of a patient A.

In some embodiments, the phased array 910 may be configured to change a width of a focal plane at the azimuthal focal point 911 relative to the phased array 910. Actively controlling the width of the azimuthal focal point 911 and making for example an azimuthal focal plane, can help improve transmission by reducing attenuation in areas of high attenuation within the body of a patient A. Every focused beam has a beam width at the azimuthal focal point 911, and in some embodiments may include a focal plane. The size of the focal plane may be changed to improve transmission characteristics through organs and tissues of the body of the patient.

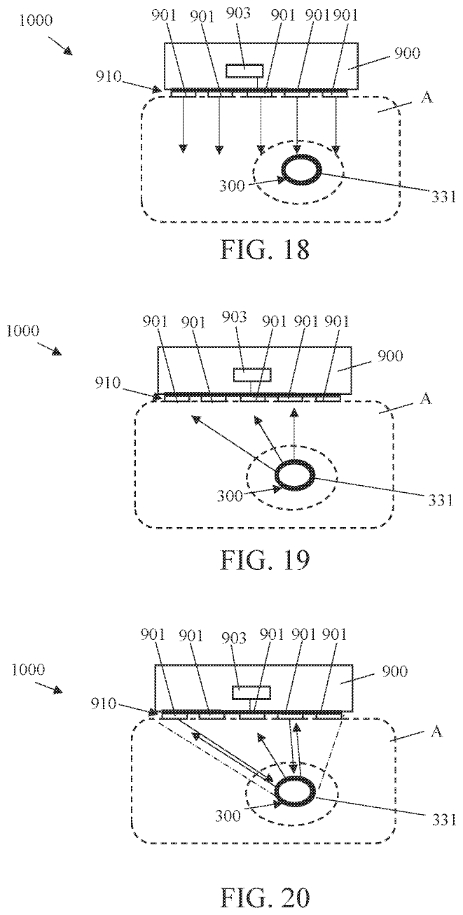

FIG. 18 shows a system 1000 for ultrasonic communication in medical implants including an implant 300 configured for ultrasonic communication and an external transceiver 900 configured to pair with and communicate with the implant 300 using ultrasound signals. The implant 300 is implanted within a patient A and shown disposed within an intramedullary canal of a bone. The implant 300 includes at least one ultrasonic transducer 331 configured to transmit and receive an ultrasound signal. An external transceiver 900 is shown configured to pair with and communicate with the implant 300 using an ultrasound signal, the external transceiver 900 includes: a phased array 910 of ultrasonic transducers 901 configured to be placed adjacent to a patient's A skin and configured to transmit and receive ultrasound signals.

The phased array 910 is operably coupled to a controller 903 and configured to rasterize one or more of the azimuthal focal point, the azimuthal focal depth and the focal width, to maximize one or more of an amount of reception of ultrasonic signals transmitted to the implant 300 and an amount of reception of ultrasonic signals received by the implant 300.

In FIG. 18, the external transceiver 900 is shown with the phased array 910 provided adjacent to the patient's skin. The external transceiver 900 may enable or awaken the implant 300 by transmitting an ultrasound signal to the implant 300, the ultrasound signal may for example may include a step function of a particular amplitude and a particular temperance configured to activate a digital switch of the implant 300. As shown, the enabling, sometimes called awakening, signal may not necessarily be focused unless for example the location of the implant 300 is known.

As shown in FIG. 19, upon awakening the implant 300 is configured to transmit an ultrasound signal to the external transceiver 900. Based on the relative location of each ultrasound transducer 901 as disposed on the phased array 910, the external transceiver 900 is configured to determine a triangulated position of the implant 300 within the body of the patient relative to the phased array 910.

As shown in FIG. 20, using the triangulated position, the external transceiver 900 may determine an optimal azimuthal focal point 911, and set the azimuthal focal point 911 accordingly to pair the external transceiver 900 with the implant 300 and achieve a maximum amount of ultrasound signal transmission.

In some embodiments, the external transducer 900 may then further rasterize the azimuthal focal point 911 in one or more of lateral position and focal depth to maximize transmission of an ultrasonic signal to the implant 300. This may be necessary because different areas of the body may have higher or lower attenuation characteristics, so by exploring different areas of transmission and paths of transmission, different signal intensities may be observed at the implant 300. Similarly, the external transducer 900 may vary a focal width of the azimuthal focal point to maximize intensity and signal quality of the ultrasonic signal at the implant 900.

The communication link may be established using serial, parallel, or any known communication protocols. And the implant 300 may communicate information corresponding to properties of the implant 300, properties observed by the implant 300, information corresponding to a biological condition, and any other information that may be useful to a physician and person skilled in the art.

The footprint of the phased array 910 may be smaller than 75×50×25 mm³. Generally, the smaller the better. The phased array 910 and even the external transducer 900 itself may be waterproof and portable. A hydrogel may be used adjacent to the phased array 910 and the skin of the patient, instead of for example liquid gel, to minimize air gap attenuation between the skin of the patient and the phased array 910.

In some embodiments, the phased array 910 may include thirty-two ultrasonic transducers 901, including a searching and tracking range configured to vary the focal depth of the azimuthal focal point from at least 25 mm to 100 mm, and laterally translate the azimuthal focal point at least +/−30 mm.

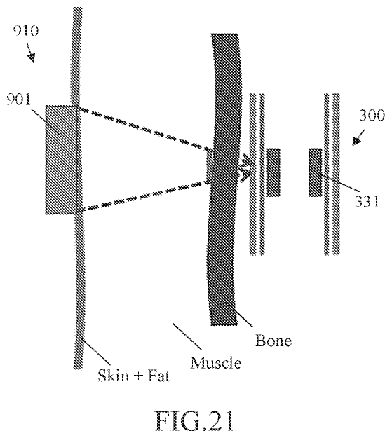

Turning to FIG. 21, the communication link loss is illustrated and estunated to be roughly 80 dB between a single ultrasonic transducer 901 and the implant 300 when transmitted through fat, bone, and a metal casing of the implant 300. The signal will experience roughly 10 Db loss through fat and muscle, roughly 30 dB loss through bone, and roughly 40 dB loss through a metal housing of the implant 310.

In some embodiments, the external transceiver 900 includes an algorithm configured to rasterize one or more of the azimuthal focal point and the azimuthal focal depth of the beam to locate the implant 300. The external transceiver 900 may be configured to report the location of the implant 300 to one or more tertiary device. The tertiary device may include a user interface and may be configured to report the location of the implant to a user.

What is claimed is:

1. An external transceiver configured for ultrasonic communication with medical implants, the external transceiver comprising:
an array of ultrasonic transducers configured to be placed adjacent to a patient's skin and each ultrasonic transducer of the array of ultrasonic transducers configured to send and receive an ultrasonic signal.

2. The external transceiver of claim 1, the array of ultrasonic transducers comprising a phased array.

3. The external transceiver of claim 2, the phased array configured to set an azimuthal focal point and steer the azimuthal focal point relative to the phased array.

4. The external transceiver of claim 3, the phased array configured to steer the azimuthal focal point laterally and change a lateral position of the azimuthal focal point relative to the phased array.

5. The external transceiver of claim 3, the phased array configured to steer the azimuthal focal point and change a focal depth of the azimuthal focal point relative to the phased array.

6. The external transceiver of claim 3, the phased array configured to change a width of a focal plane at the azimuthal focal point relative to the phased array.

7. A system for ultrasonic communication in medical implants, the system comprising:
an implant configured to be implanted within a patient, the implant comprising an ultrasonic transducer configured to transmit and receive ultrasonic signals; and
an external transceiver configured to communicate with the implant using an ultrasound signal, the external transceiver comprising: an array of ultrasonic transducers configured to be placed adjacent to a patient's skin and configured to transmit and receive ultrasonic signals.

8. The system of claim 7, the array of ultrasonic transducers comprising a phased array.

9. The external transceiver of claim 8, the phased array configured to set an azimuthal focal point and steer the azimuthal focal point relative to the phased array.

10. The external transceiver of claim 9, the phased array configured to steer the azimuthal focal point laterally and change a lateral position of the azimuthal focal point relative to the phased array.

11. The external transceiver of claim 9, the phased array configured to steer the azimuthal focal point and vary a focal depth of the azimuthal focal point relative to the phased array.

12. The external transceiver of claim 9, the phased array configured to change a width of a focal plane at the azimuthal focal point.

13. The system of claim 9, the phased array configured to rasterize the azimuthal focal point to maximize an amount of transmission of ultrasonic signals transmitted to the implant.

14. The system of claim 9, the phased array configured to rasterize a focal depth of the azimuthal focal point to maximize transmission of the ultrasonic signal transmitted to the implant.

15. A method for ultrasonic communication in medical implants, the method comprising the steps:
implanting an implant within a patient, the implant comprising an ultrasonic transducer configured to transmit and receive ultrasonic signals;
providing adjacent to the patient's skin an external transceiver configured to communicate with the implant using an ultrasound signal, the external transceiver comprising: an array of ultrasonic transducers configured to transmit and receive ultrasonic signals.

16. The method of claim 15, comprising the step:
transmitting an ultrasound signal to the implant using the external transceiver, the ultrasound signal configured to activate the implant.

17. The method of claim 16, the phased array configured to set an azimuthal focal point and steer the azimuthal focal point relative to the phased array.

18. The method of claim 17, comprising the step:
rasterizing a lateral position of the azimuthal focal point to maximize transmission of an ultrasonic signal transmitted to the implant.

19. The method of claim 17, comprising the step:
rasterizing a focal depth of the azimuthal focal point to maximize transmission of an ultrasonic signal transmitted to the implant.

20. The method of claim 17, comprising the step:
varying a focal width of the azimuthal focal point to maximize transmission of an ultrasonic signal transmitted to the implant.
